(19) United States
(12) Patent Application Publication
(10) Pub. No.: US 2005/0205088 A1
(43) Pub. Date: Sep. 22, 2005

(54) Nebulizer mouthpiece

(76) Inventors: Tuan Q. Tran, Richmond, VA (US); Lawrence A. Weinstein, Chesterfield, VA (US)

Correspondence Address: McGuire Woods LLP;Suite 1800, 1750 Tysons Boulevard, McLean, VA, 22102, US

(21) Appl. No.: 10/802,745

(22) Filed: Mar. 18, 2004

(51) Int. Cl. A61M 11/00; B05B 17/06; A62B 7/00

(52) U.S. Cl. 128/200.24

(57) ABSTRACT

A mouthpiece which is attachable to nebulizer is configured to enhance the flow of air through an aerosolization portion of the nebulizer. The mouthpiece has a hollow body attached to a drug delivery conduit. The drug delivery conduit has a drug delivery port in one end. The interior of the hollow body is configured to efficiently direct airflow into the aerosolization chamber of the nebulizer to allow better mixing of the aerosolized substance and the air before the air exits the nebulizer. The mouthpiece is also configured for ease of use as well as being configured to attach to a variety of debulization chambers, and may include a vent having one or more diaphragms to direct airflow along center flow paths.

### BACKGROUND OF THE INVENTION

1. Field of the Invention

The invention relates to a drug delivery device, and more particularly to a nebulizer mouthpiece for delivering drugs to a patient.

2. Background Description

Drugs and other substances can be administered to a patient through a variety of methods. For example, one method includes intravenous injection where the drug or substance is injected into a person's veins. Another method includes a sublingual delivery system where the drug or substance is placed under the user's tongue and is allowed to be absorbed through the mucus membranes of the mouth. Still another method of drug or substance delivery includes applying the drug or substance directly onto the recipients skin where it either treats a condition on the surface of the skin or is absorbed into the body.

One method of administering a drug or substance to a patient suffering from respiratory as well as other conditions includes inhaling air or a gas in which a drug or other substance is dissolved, mixed, entrained, suspended or otherwise incorporated into the air or gas. With this delivery method, the air or gas functions as a vehicle which carries the drug or substance into the user's lungs where is may be absorbed to treat a condition of the lungs or in some instances be transferred into the circulation system of the user to be distributed throughout the body.

It should be noted that although a drug is typically the material which is delivered in this way, virtually any chemical or substance either in a gaseous, liquid, or solid form may be delivered using this inhalation technique. Furthermore, the vehicle which is typically air may also be made up only of, or partially of, any kind of gas or mixture of gases where the gas or mixture of gases serves to not only transport the drug or substance into the users lungs, but may also interact with the users tissues as well as interact with the drug or substance being delivered. Such interaction may further enhance the chemical or medical activity of the drug or substance, as well as suppress or enhance certain responses of the tissue to the drug or substance to delivered.

The drug or substance to be delivered can be incorporated into the vehicle, which will be referred to as air hereinafter for simplicity, through a variety of methods. For example, the drug can take the form of a gas and be mixed into the air and remain suspended in the air, or dissolved in the air. Additionally, the delivered substance may exist initially in the form of a bulk liquid and be converted to small droplets or a mist which is mixed or entrained into the air for transport into the lungs. Furthermore, the delivered substance may be converted from a liquid into a vapor through heating, for example, for incorporation into the air. Also, the delivered substance may exist in solid form as particles which are mixed, entrained, or otherwise suspended in the air while the air is inhaled by the user.

Because the delivered substance may exist in a variety of forms all of which have different degrees of ease of incorporation into the air flowing into a user's lungs, an efficient means for incorporating the delivered substance into the air is needed. A typical way of incorporating the delivered substance into air is an aerosolization chamber, or nebulizer.

Additionally, the delivered substance can exist in a first form which is then mixed with a carrier substance such as mixing solid particles into a liquid and the resulting liquid-particle mixture is then converted into a mist and incorporated into the air.

Typically, a nebulizer uses the flow of air through a chamber to cause turbulence between the air and the substance at their interface in order to form a mixture of the substance and air, the delivered substance is then carried into the users lungs along a flow path from the mixing area to the user's mouth or nose.

Accordingly, mouthpiece is typically joined to the nebulizer which is then inserted into the patient's mouth during inhalation through the mouth. As such, as the user inhales, air is drawn through the mouthpiece and through the nebulizer which initiates the mixing of the delivered substance in the air.

Because some delivered substances may be difficult to incorporate into air, and other delivered substances must be delivered at dosage rates which require the air to carry a large amount of the delivered substance, it is important that the nebulizer and mouthpiece work to efficiently mix the delivered substance into the air. Furthermore, because some patients being treated using this inhalation technique are in a weakened or otherwise impaired condition and cannot inhale air with a force sufficient to power the mixing process in the nebulizer, efficient mixing is advantageous.

For the above stated reasons, a mouthpiece is needed which can be attached to a variety of nebulizers which makes efficient use of air passing through the mouthpiece and nebulizer to easily incorporate a delivered substance into the air flow. Furthermore, a mouthpiece which directs air into and out of a nebulizer in such a way as to efficiently mix a delivered substance into the inhaled air is needed. Also, a mouthpiece which aids in efficiently mixing a delivered substance into air and a nebulizer, and which will attach to a variety of nebulizer styles from various manufacturers is needed.

### SUMMARY OF THE INVENTION

In a first aspect of the invention, the drug delivery system includes a top flow path, and a conduit with an end in communication with the top flow path and another end configured to communicate with a nebulizer. The system also includes a nebulizer in-flow path disposed within the conduit configured to communicate with a nebulizer, and a nebulizer out-flow path disposed within the conduit configured to communicate with a nebulizer and the top flow path, wherein the nebulizer out-flow path is separate from the nebulizer in-flow path.

In another aspect of the invention, the drug delivery system includes a nebulizer mouthpiece, having a user interface having a delivery port and a conduit connected to the user interface, wherein the user interface and the conduit are configured to define a flow path between an aerosolization area and the delivery port. The nebulizer mouthpiece also includes an intake flow path configured to communicate with an aerosolization area, and an exhaust flow path configured to communicate with an aerosolization area, wherein the exhaust flow path and the intake flow path are combined.

In yet another aspect, the invention includes a drug delivery mouthpiece having a hollow body with a top and bottom, and an intake port and a delivery port proximate the top of the hollow body. The invention also includes a nebulizer inlet and nebulizer outlet proximate the bottom of the hollow body, and a baffle at least partially disposed within the hollow body.

### BRIEF DESCRIPTION OF THE DRAWINGS

FIG. 1 is a illustration of an embodiment of the invention;

FIG. 2 is a cutaway view of an embodiment of the invention;

FIG. 3 is a side view of an embodiment of the invention;

FIG. 4 is a ghost view of an embodiment of the invention;

FIG. 5 is a bottom view of an embodiment of the invention;

FIG. 6 is a front view of an embodiment of the invention;

FIG. 7 is a quarter view of an embodiment of the invention;

FIG. 8 is a rear view of an embodiment of the invention;

FIG. 9 is a rear view of an embodiment of the invention;

FIG. 10 is a quarter view of an embodiment of the invention;

FIG. 11 is a bottom view of an embodiment of the invention;

FIG. 12 is a top view of an embodiment of the invention;

FIG. 13 is a ghost view of an embodiment of the invention;

FIG. 14 is a front quarter view of an embodiment of the invention;

FIG. 15 is a rear quarter view of an embodiment of the invention;

FIG. 16 is a bottom view of an embodiment of the invention;

FIG. 17 is a top view of an embodiment of the invention; and

FIG. 18 is a rear view of an embodiment of the invention.

### DETAILED DESCRIPTION OF EMBODIMENTS OF THE INVENTION

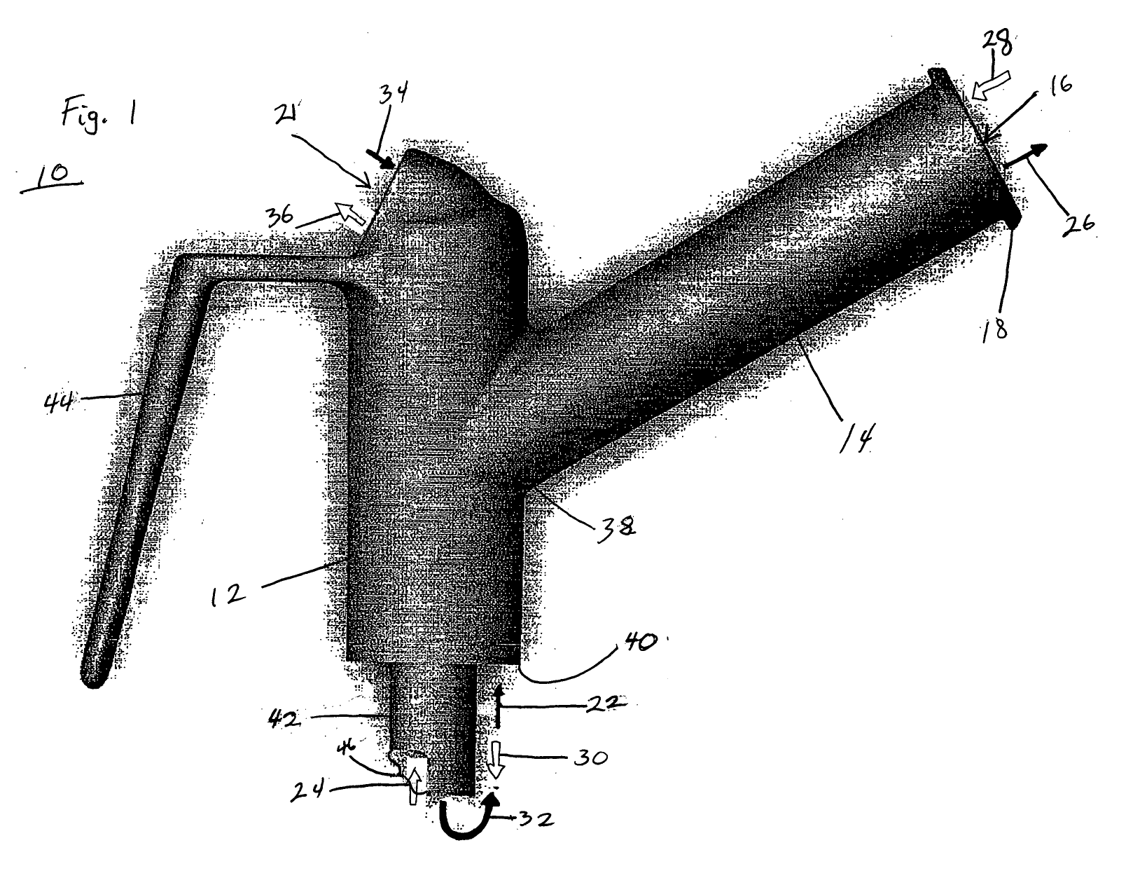

Referring now to the drawings, FIG. 1 is an illustration of an embodiment of a breath enhanced mouthpiece 10. The breath enhanced mouthpiece 10 includes a hollow body 12 also referred to as a body conduit. Attached to the hollow body 12 is a drug delivery conduit 14, also referred to as a drug conveyance. The hollow body 12 and the drug delivery conduit 14 are attached to one another at an intersection 38, also referred to as a union or junction. The drug delivery conduit 14 also includes a user interface 18 which defines a drug delivery port 16 at an end of the drug delivery conduit 14. The drug delivery port 16 can also be referred to as an orifice, opening or aperture, etc.

The drug delivery port 16 defines both an outlet and an inlet into a top flow path. The top flow path at the drug delivery port 16 can be defined as an outflow path 26 upon inhalation and an inflow path 28 upon exhalation, both moving air through the same volume of drug delivery conduit 14 and through the drug delivery port 16 at different times depending upon whether the patient is inhaling or exhaling.

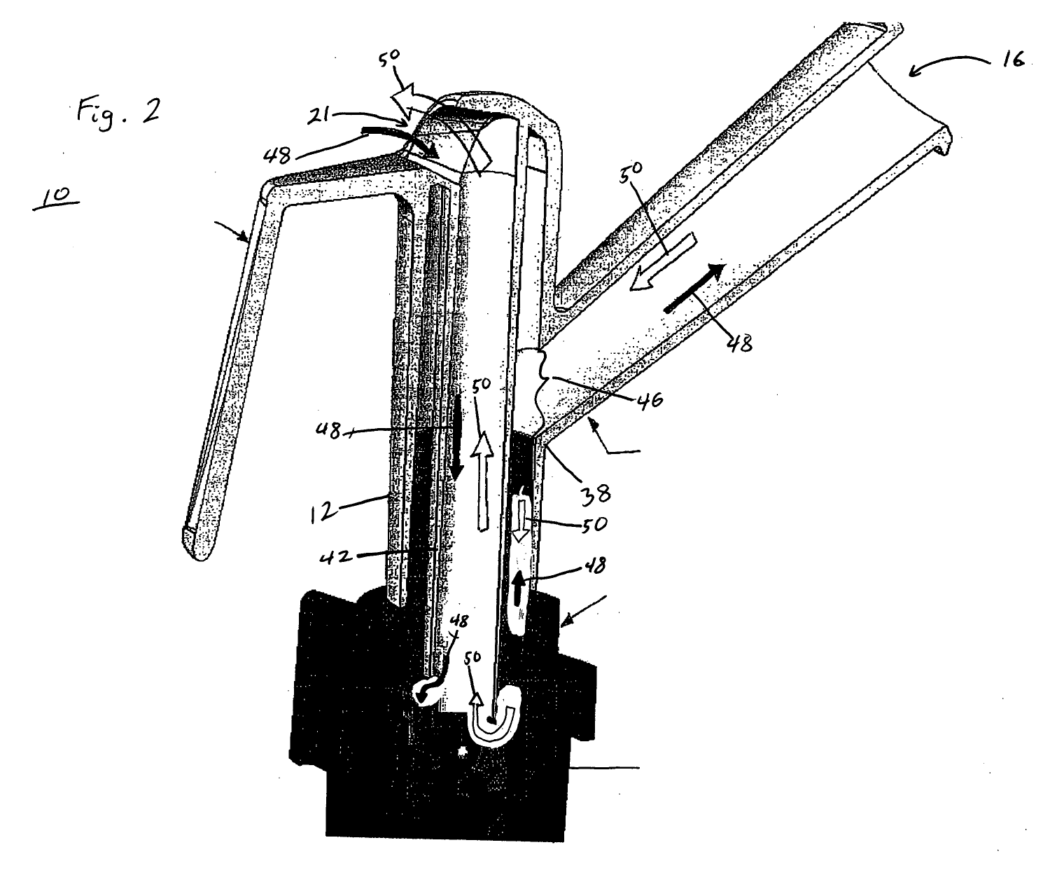

The hollow body 12 has a connection port 40 or opening at its lower end. Within the hollow body 12 is a baffle 42 which can either be planer, circular, tubular, etc. Together, the hollow body 12 and the baffle 42 define two air flow paths within the hollow body 12. For example, in FIG. 2 the baffle 42 is a tubular conduit. Accordingly, one air flow path is through the interior of the baffle 42 and another air flow path is along the exterior of the baffle 42. Both of the aforementioned air flow paths are through the hollow body 12 of the breath enhanced mouthpiece 10. An end of the baffle 42 may include a notch or cut out 46.

More specifically, the airflow path through the hollow body 12 may be subdivided into sections. Accordingly, upon inhalation, the air flow path through the interior of the baffle 42 may include an intake flow path 32, also referred to as a nebulizer inflow path. The air flow path 32 within the baffle 42 may also include an exhaust air flow path 24 which occupies the same volume within the baffle 42 upon exhalation. The baffle 42 and the hollow body 12 also define an air flow path therebetween which include a drug delivery flow path 22, also referred to as an outflow path. Also included in the same volume as this air flow path is an exhaust flow path 30.

The hollow body 12 has a top which includes a vent 21. The vent 21 may be configured to be selectively sealable. The vent 21 defines one or more air flow paths through the top of the hollow body 12 including an intake flow path 34 and an exhaust flow path 36. Where the baffle 42 is a conduit, the vent 21 may communicate with an end of that conduit. The hollow body 12 may also include a handle 44 configured to aid in grasping the breath enhanced mouthpiece 10.

In this embodiment of the breath enhanced mouthpiece 10, the drug delivery conduit 14 joins the hollow body 12 at an angle of less than 90 degrees as referenced to the top of the hollow body 12. It should be noted that the drug delivery conduit 14 may join the hollow body 12 at substantially any angle desired, and is preferably configured to be joined at an angle configured for ease of insertion into a user's mouth.

Thus the entire flow path upon inhalation includes flow paths of 22, 26, 32, and 34 of FIG. 1. The entire flow path upon exhalation includes flow paths 28, 30, 24, and 36 of FIG. 1.

As shown in FIG. 1, the mouthpiece 10 is configured to easily attach and detach to other fittings, such as, for example, an aerosolization or nebulization chamber. Accordingly, the mouthpiece 100 may be fitted and removed from a aerosolization chamber at the connection port 40, and thus can be used on a variety of aerosolization chamber, as well as any other type of fitting which may attach at the connection port 40. Thus, although the connection port 40 is shown as a reversible slip or press-fit type of connection, the connection port 40 may be configured to attach by any method well known in the art which allows a detachable or reversible connection to be made, such as threads, a clamp, or a lock.

Referring to FIG. 2, the breath enhanced mouthpiece 10 is shown in a cut away view showing air flow paths. An entire intake air flow path 48 shown unsubdivided is represented by the solid arrows and travels from the vent 21 near the top of the hollow body 12 through the interior of the baffle 42 to exit out the bottom of the baffle 42 proximate an aerosolization area. After passing through the aerosolization area, the intake flow path 48 continues between the baffle 42 and the wall of the hollow body 12 to the union 38 between the hollow body 12 and the drug delivery conduit 14.

As shown in FIG. 2, the walls of the hollow body 12 and the walls of the drug delivery conduit 14 define a union inlet 46 at the union 38. The intake flow path 48 passes through the union inlet 46 and proceeds through the drug delivery conduit 14 towards the drug delivery port 16. The intake flow 48 exits the breath enhanced mouthpiece 10 through the drug delivery port 16.

The breath enhanced mouthpiece 10 also defines a second air flow path also referred to as an exhaust air flow path 50 represented by the hollow, arrows. It should be noted the exhaust air flow path 50 may flow along the same volume of flow as the intake flow path 48, however, in the opposite direction thereto and at different times during use of the mouthpiece 10. Accordingly, the exhaust air flow path 50 flows along a path from the drug delivery port 16 down through the interior of the drug delivery conduit 14 and through the union inlet 46. The exhaust flow path 50 enters the hollow body 12 and flows through the hollow body 12 along the outside of the baffle 42 towards the bottom of the baffle 42. The exhaust flow 50 then enters the bottom of the baffle 42 and flows towards the top of the hollow body 12. Proximate the top of the baffle 42 and hollow body 12, the exhaust flow path 50 exits the hollow body 12 through a vent 21. As can be seen, the intake flow path 48 and the exhaust flow path 50 are substantially along the same path, but opposite of one another.

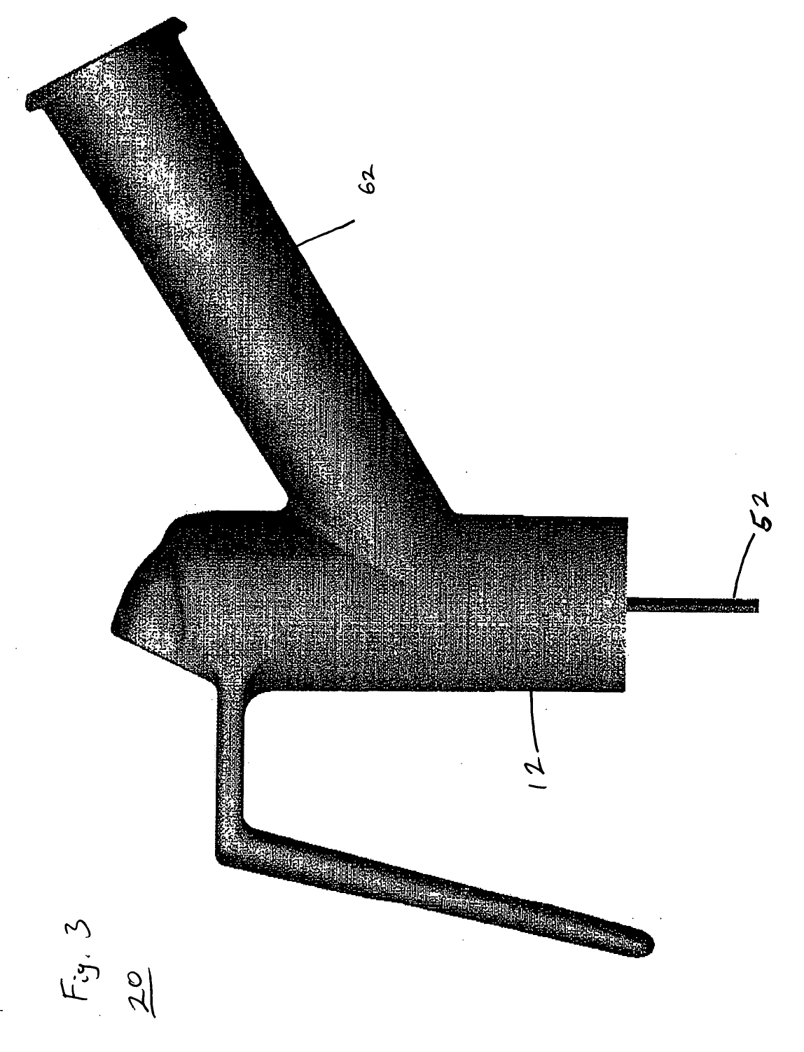

Referring to FIG. 3, another embodiment of the invention is shown as a breath enhanced mouthpiece 20. This embodiment of the breath enhanced mouthpiece 20 has a flat or planer baffle 52 in place of the conduit-like baffle 42 of FIG. 1. As such, the hollow body 12 and the flat baffle 52 define an air flow path having portions parallel to one another through the interior of the hollow body 12. The baffle 52 may protrude from the bottom of the hollow body 12 with one of its planer surfaces facing towards the drug delivery conduit 14.

As shown in FIG. 4, the flat baffle 52 and the hollow body 12 define a flow path through the hollow body 12. The flow path extends from a vent 58 at the top of the hollow body 12 and through the interior of the hollow body 12 mounted on one side by the flat baffle 52. The flow path 54 passes around the bottom of the flat baffle 52 and along the opposite side of the flat baffle 52 back into the interior of the hollow body 12. The flow path 54 then passes through the union inlet 56 and into a drug delivery conduit 62 to exit out a drug delivery port 60.

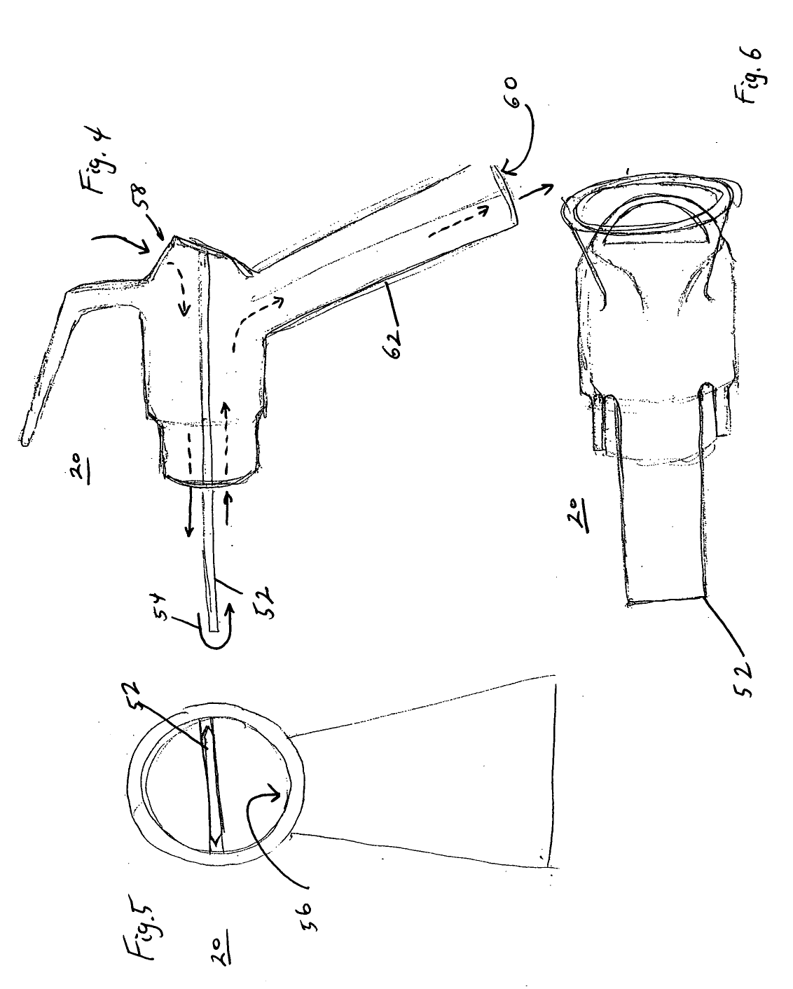

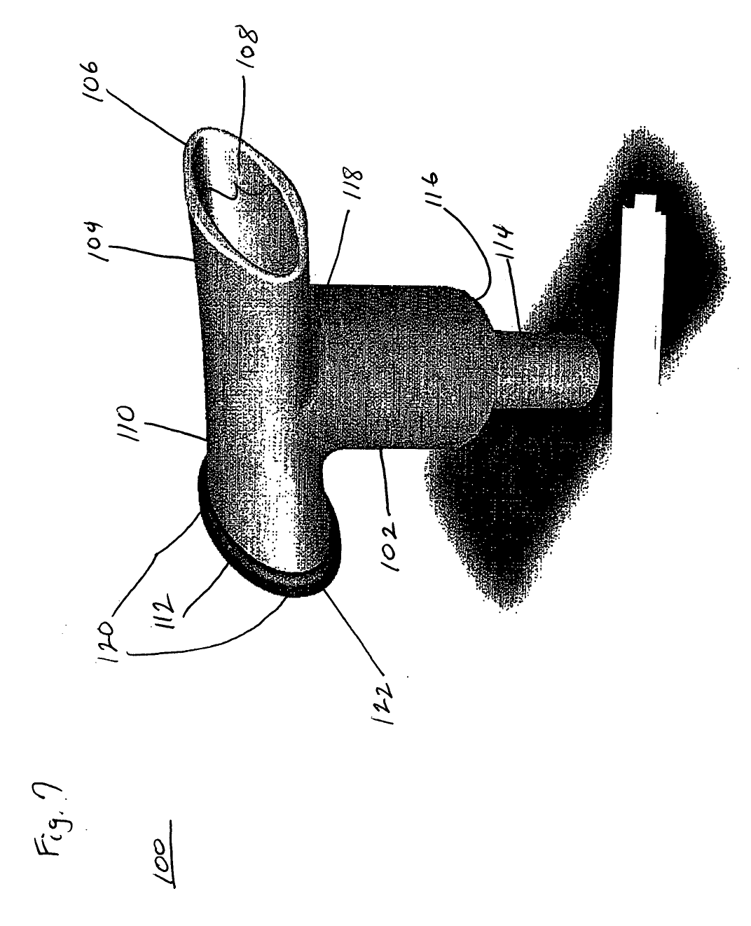

Referring to FIG. 5, a bottom view of the breath enhanced mouthpiece 20 is shown which illustrates how the flat baffle 52 divides the cavity within the hollow body 12 into a first half and a second half where one of the halves contains a union inlet 56 of the breath enhanced mouthpiece 20.

Referring to FIG. 6, the breath enhanced mouthpiece 20 has the flat baffle 52 protruding out of the bottom of the hollow body 12 and has a substantially flat bottom terminating the flat baffle 52. It should be noted that the flat baffle 52 and bottom of the baffle may take on any shape which enhances the efficiency of the mixing process in an attached nebulizer.

Referring to FIG. 7, a further embodiment of the breath enhanced mouthpiece 100 is shown. This embodiment of the breath enhanced mouthpiece 100 has a hollow body 102 coupled to a drug delivery conduit 104 and a vent conduit 110. The hollow body 102 is joined to the drug delivery conduit 104 and the vent conduit 110 at a junction 118. The junction 118 can also be referred to as a union or an intersection.

The drug delivery conduit 104 includes a user interface 106 and an opening which defines a drug delivery port 108. Vent conduit 110 includes a selectively sealable vent 112 on an end. The hollow body 102 includes a connection port 116 at a lower end of the hollow body 102. Disposed within the hollow body 102 and protruding from the hollow body 102 through the drug connection port 116 is a baffle 114. Although FIG. 7 shows the baffle 114 being circular or tubular, the baffle 114 can be of virtually any shape sufficient to guide a flow of air including a flat or planar structure. In the embodiment in FIG. 7, the baffle 114 forms a tubular conduit which defines a flow path therein.

The breath enhanced mouthpiece 100 also includes multiple vents on an end of the vent conduit 110. The vent includes a selectively sealable inlet vent 112 and selectively sealable outlet vents 120. Also included on the end of the vent conduit 110 is a diaphragm retainer 122 which is configured to hold a diaphragm on the end of the vent conduit 110.

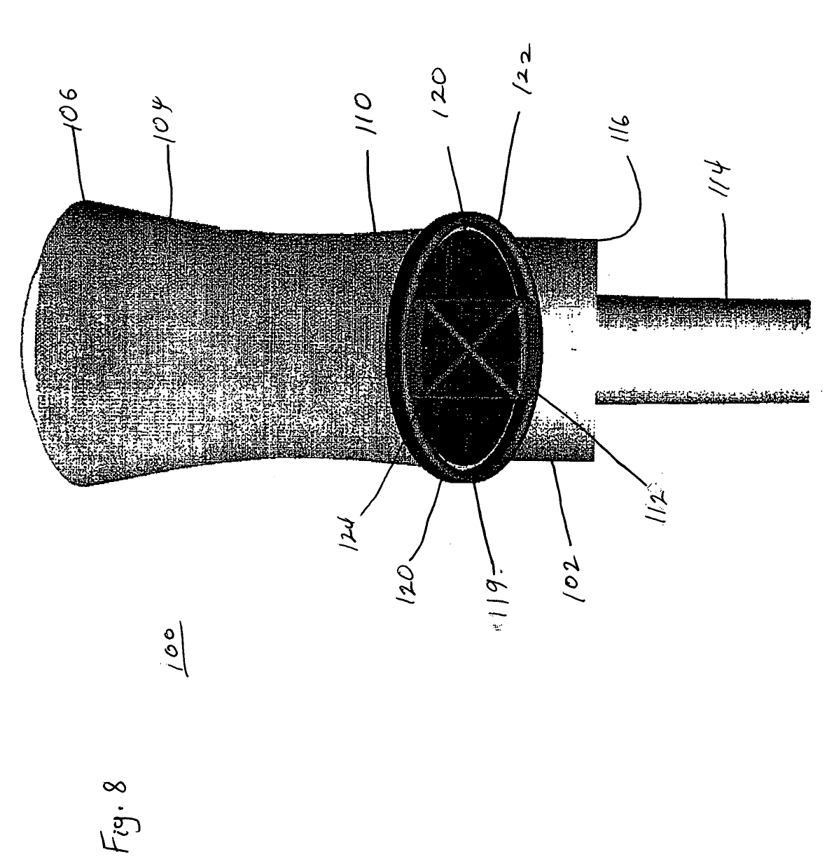

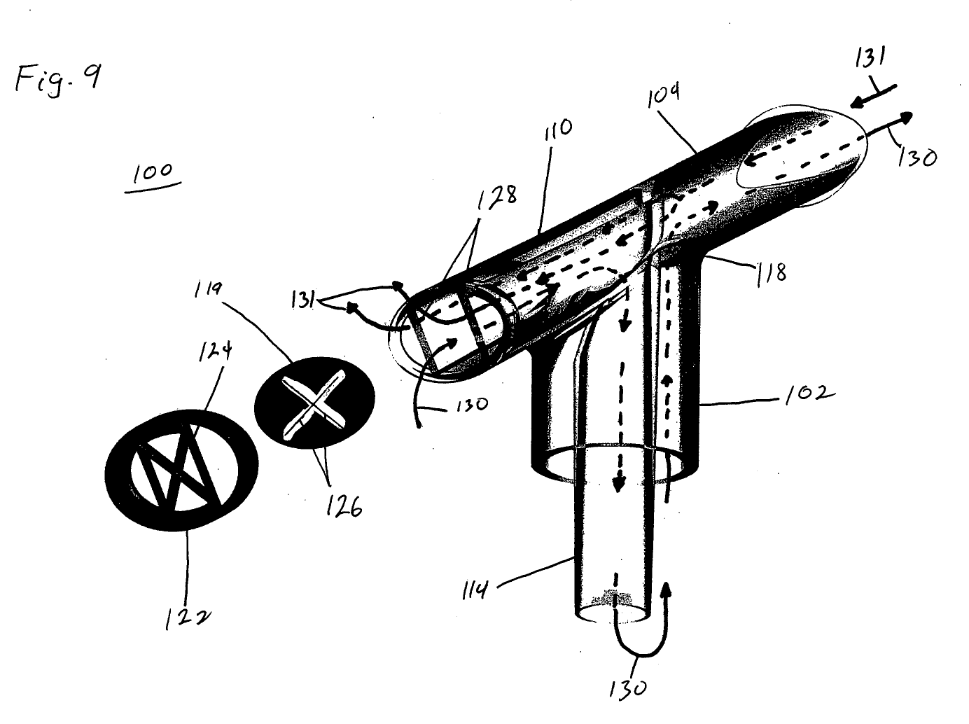

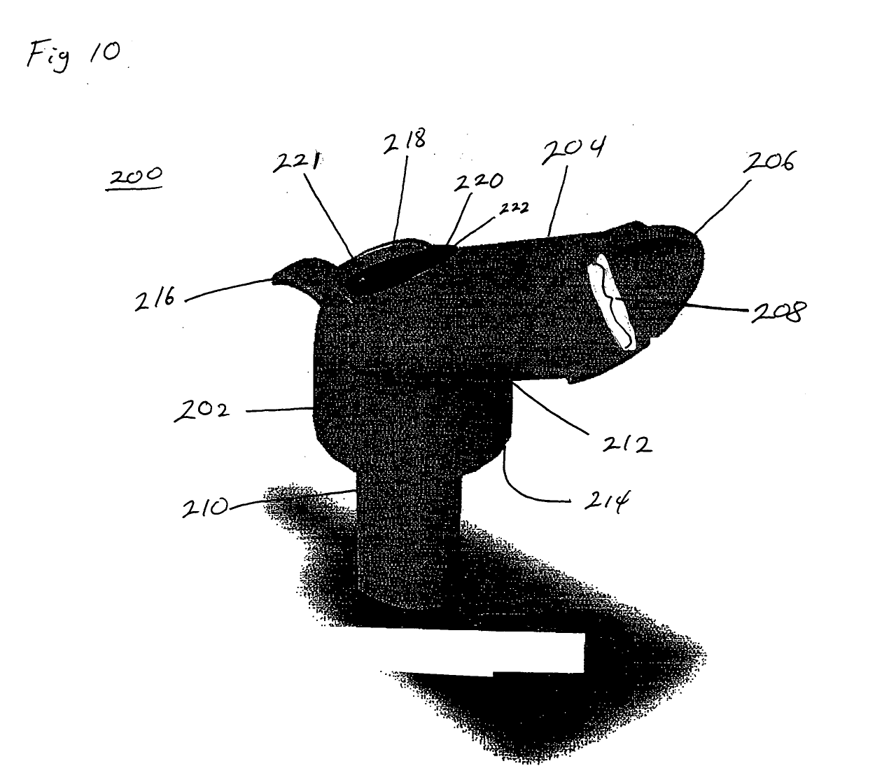

Referring to FIG. 8, a rear view of the breath enhanced mouthpiece 100 is shown. The rear view shows the selectively sealable inlet vent 112 and the selectively sealable outlet vents 119 on either side of the inlet vent 112 on the end of the vent conduit 110. Also shown is the diaphragm retainer 122 and the diaphragm 120. The diaphragm retainer 122 has a diaphragm support 124 which is configured to lie flat across the diaphragm 119. The diaphragm retainer 122 is configured to hold the diaphragm 119 over the end of the vent conduit 110.

Referring to FIG. 9, an exploded view of the breath enhanced mouthpiece 100 is shown. The vent conduit 110 has walls 128 therein, which divide the interior of the vent conduit 110 into multiple flow paths. The diaphragm 119 is configured to fit over the end of the vent conduit 110 and has slits 126 formed therein. The diaphragm 119 may also have other types of opening beside the slits such as an orifice, aperture, flap, etc. The diaphragm retrainer 122 is configured to fit over the end of the vent conduit 110 and hold the diaphragm 119 in place on the end of the vent conduit 110. As can be seen, the diaphragm retainer 122 has a diaphragm support 124 which are portions of the diaphragm retainer 122 which approximately correspond to the slits 126 in the diaphragm 119.

The structure of the breath enhanced mouthpiece 100 is configured to define various air flow paths therein. For example, one of the flow paths includes a delivery flow path 130. The delivery flow path 130 may begin at the selectively sealable inlet vent 112 and pass between both walls 128 in the interior of the vent conduit 110. Proximate the union 118, the delivery flow path 130 turns and enters the baffle 114 and passes through the interior of the baffle 114 and out the baffle's lower end. At this point, the delivery flow path 130 may pass through an aerosolization or nebulizing area where drugs or other substances in the forms a gas, a vapor, or small particles or droplets may be entrained, mixed or otherwise incorporated into the air passing along the flow path 130. The delivery flow path 130 then proceeds along the exterior of the baffle 114 towards the union 118. The delivery flow path 130 enters the drug delivery conduit 104 proximate the union 118, and proceeds along the drug delivery conduit 104 to exit therefrom through the drug delivery port 108.

The breath enhanced mouthpiece 100 includes other flow paths through its interior. For example, an exhaust flow path 131 may include a path from the drug delivery port 108 and through the drug delivery port 104. Proximate the union 118, the exhaust flow path 131 passes into the vent conduit 110 and divides into two paths. Each path passes between a wall 128 and the interior wall of the vent conduit 110. The exhaust flow path 131 then exits the breath enhanced mouthpiece 100 through the selectively sealable output vents 120 past the diaphragm 119.

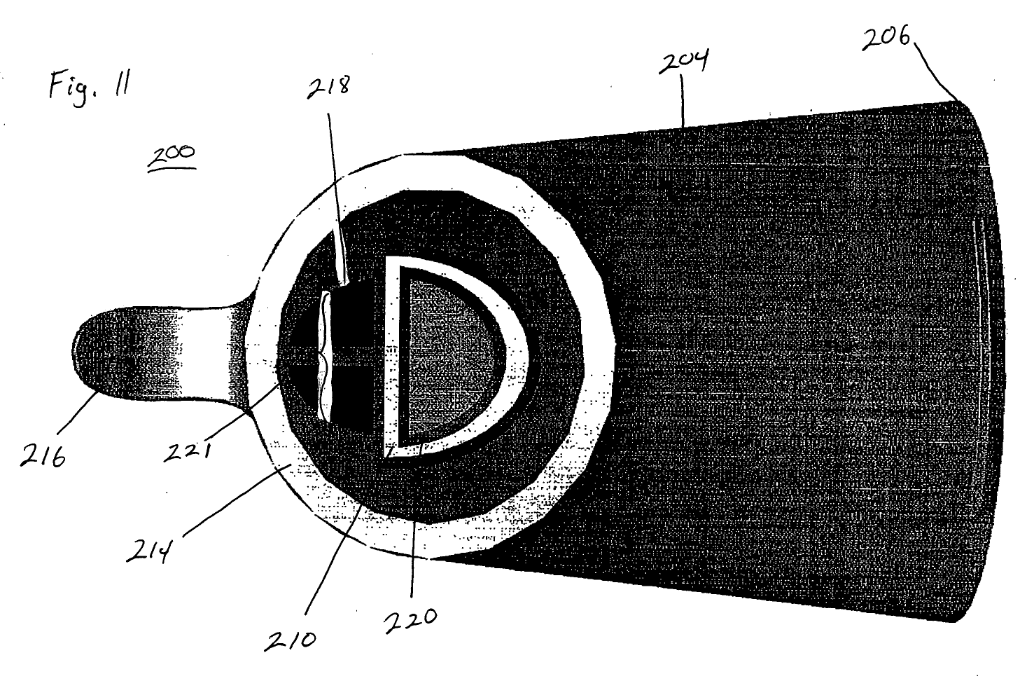

Referring to FIG. 10, a further embodiment of a breath enhance mouthpiece 200 is shown. The breath enhanced mouthpiece 200 includes a hollow body 202 connected to a drug delivery conduit 204 at a junction or union 212. The hollow body 202 includes a connection port 214 proximate its lower portion. Disposed within the hollow body 202 and protruding from the bottom of the hollow body 202 is a baffle 210 which defines a conduit within the baffle.

The drug delivery conduit 204 includes a mouthpiece 206 and a port 208 at an end of the drug delivery conduit 204. Proximate to where the hollow body 202 and drug delivery conduit 204 join at the union 212, is an exhaust aperture 221 covered by a diaphragm 218. The exhaust aperture 221 is located near the top of the hollow body 202. On the exterior of the hollow body 202 near the exhaust aperture 221 is a protrusion 216, which may be used as a hook by which to support the breath enhanced mouthpiece 100 as well as aid a user in holding the breath enhanced mouthpiece 200 during inhalation.

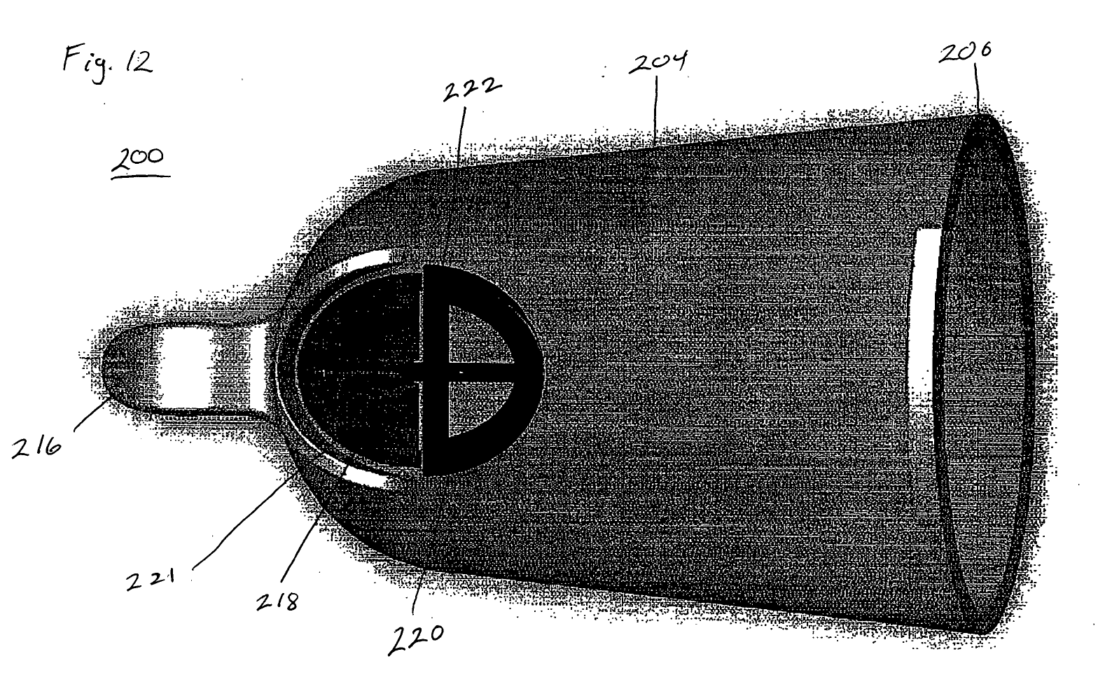

Referring to FIG. 11, a bottom view of the breath enhanced mouthpiece 200 is shown. The bottom of the hollow body 202 terminates in a connection port 214. Disposed within the hollow body 202 is a baffle 210 which, in this example, is a hollow conduit with a perimeter that, defines an approximate “D” shape in cross section. The baffle 210 is attached to the interior of the hollow body 202 near the top of the hollow body 202. Consequently, the baffle 210 is attached to the hollow body 202 proximate the aperture exhaust 221.

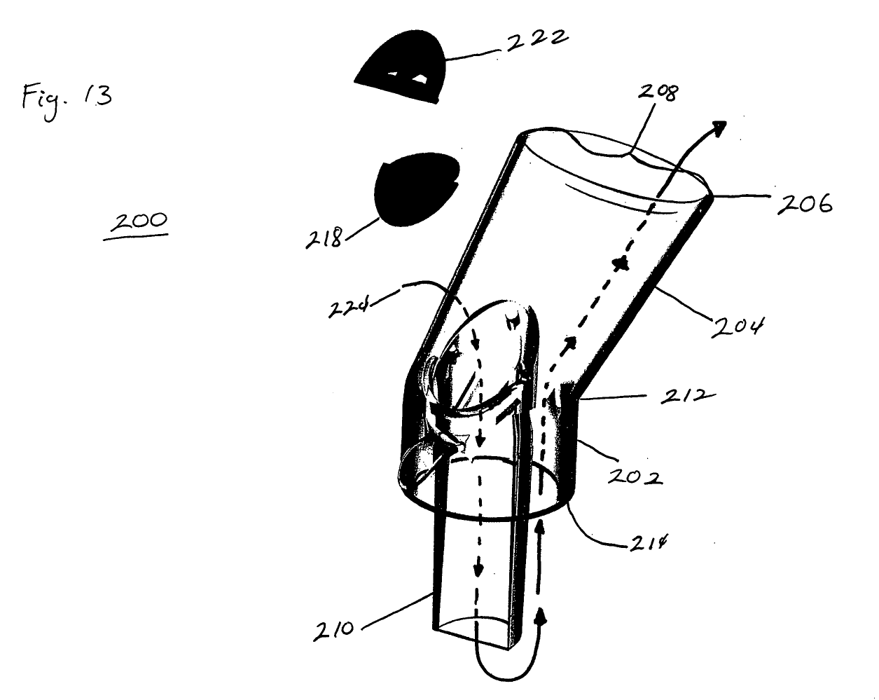

Referring to FIG. 12, a top view of the breath enhanced mouthpiece 200 is shown. Visible on the top of the breath enhanced mouthpiece 200 is the exhaust aperture 221 and an intake aperture 220. The exhaust aperture 221 is covered by a diaphragm 218 and communicates between the exterior of the breath enhanced mouthpiece 200 and the interior of the hollow body 202. The intake aperture 220 is disposed in the breath enhanced mouthpiece 200 proximate to where the baffle 210 attaches to the hollow body 202. Accordingly, the intake aperture 220 allows communication between the exterior of the breath enhanced mouthpiece 200 and the interior of the baffle 210.

The diaphragm 218 is fixed over the exhaust aperture 221 and the intake aperture 220. It should be noted that in this particular embodiment, the intake aperture 220 is configured so that air may flow from outside the breath enhanced mouthpiece 200 and through the intake aperture 220 into the interior of the hollow body 202, and in particular, into the interior of the baffle 210. The exhaust aperture 221 is also configured so that air may flow from inside the hollow body 202 and through the exhaust aperture 221 to the exterior of the breath enhanced mouthpiece 200.

The breath enhanced mouthpiece 200 also includes a drug delivery conduit 204 with a user interface 206. A protrusion 216 for storage or to facilitate mouthpiece use is attached to the hollow body 202 near the exhaust aperture 221.

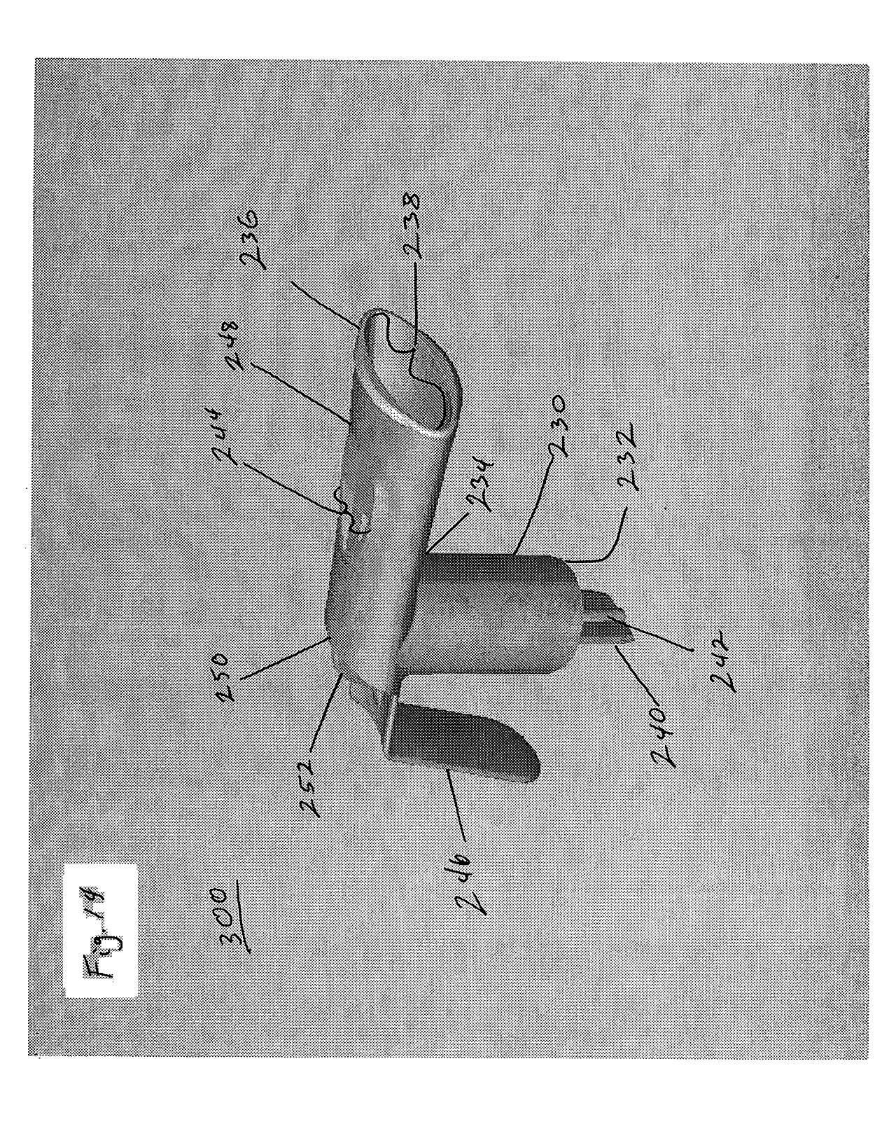

Referring to FIG. 13, the breath enhanced mouthpiece 200 is shown in ghost view. A delivery flow path 224 enters the breath enhanced mouthpiece 200 through the intake aperture 220. From the intake aperture 220, the delivery flow path 224 proceeds along the interior of the baffle 210. The delivery flow path 224 exits the baffle 210 near the bottom of the baffle 210 and proceeds along the exterior of the baffle 210 towards the union 212. At the union 212, the delivery flow path 224 enters the drug delivery conduit 204. The delivery flow path 224 proceeds along the interior of the drug delivery conduit 204 and exits the breath enhanced mouthpiece 200 at the drug delivery port 208.

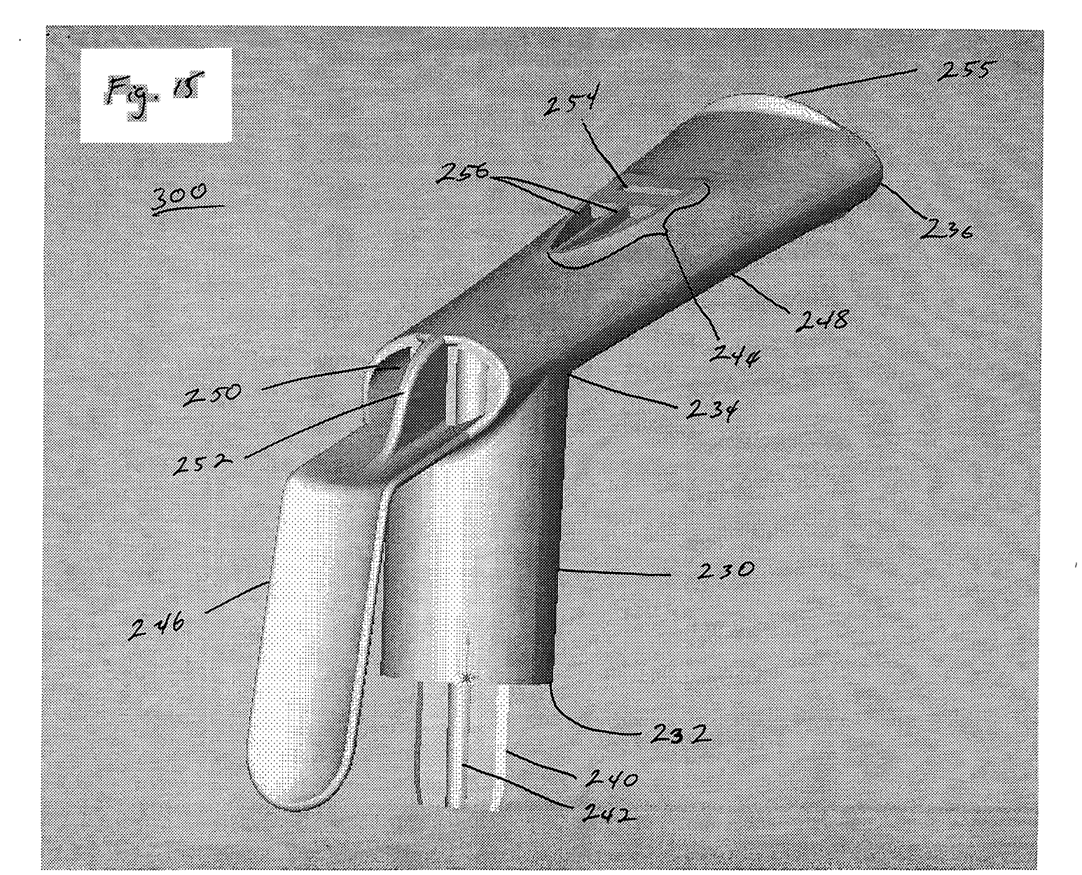

Referring to FIG. 14, another embodiment of the breath enhanced mouthpiece 300 is shown. The breath enhanced mouthpiece 300 includes a hollow body 230 attached to a drug delivery conduit 248 at a union 234. The hollow body 230 has a connection port 232 at its lower end and contains a baffle 240 running along a length of the interior of the hollow body 230. In this example, the baffle 240 is approximately planar with a ridge-like structure 242 running along the baffle's length.

The drug delivery conduit 248 has a mouthpiece 236 at one end and an inflow vent 250 at the opposite end. The mouthpiece 236 may also be referred to as a user interface. At the mouthpiece 236 is a drug delivery port 238 which is an opening which leads into the interior of the drug delivery conduit 248. On the top of the drug delivery conduit 248 is an exhaust vent structure 244. The inflow vent 250 includes a guard 252 configured to prevent objects such as, for example, a user's fingers from obstructing the inflow vent 250 during use. Proximate the guard 252 is attached a handle 246.

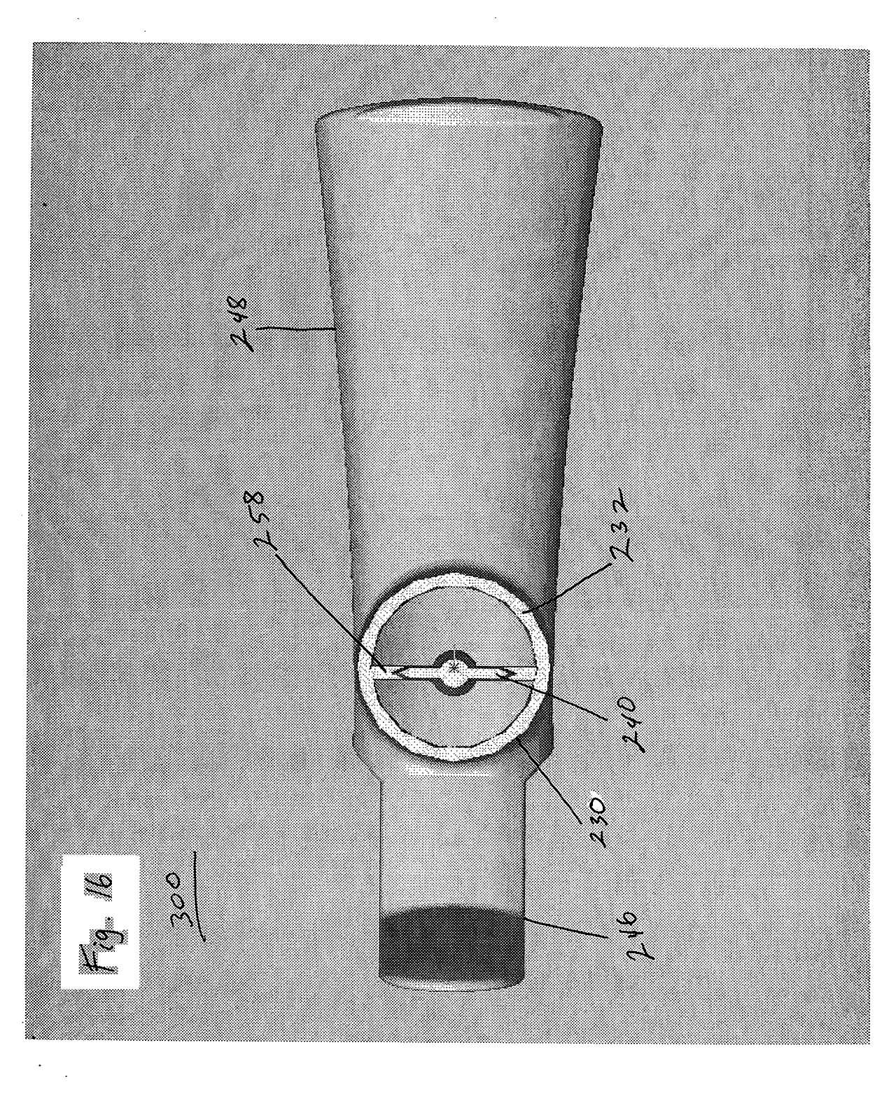

Referring to FIG. 15, the exhaust vent structure 244 includes two outlets 256, and a recess or aperture 254. The recess 254 is configured to attach a diaphragm to the top of the drug delivery conduit 248 which will selectively seal the two outlets 256. The recess 254 may also be an aperture leading into the interior of the drug delivery conduit 248. The breath enhanced mouthpiece 300 also includes ridges 255 on the drug delivery conduit 248 proximate the drug delivery port 236. The ridges 255 are configured to facilitate a user holding the mouthpiece 300 in his or her mouth.

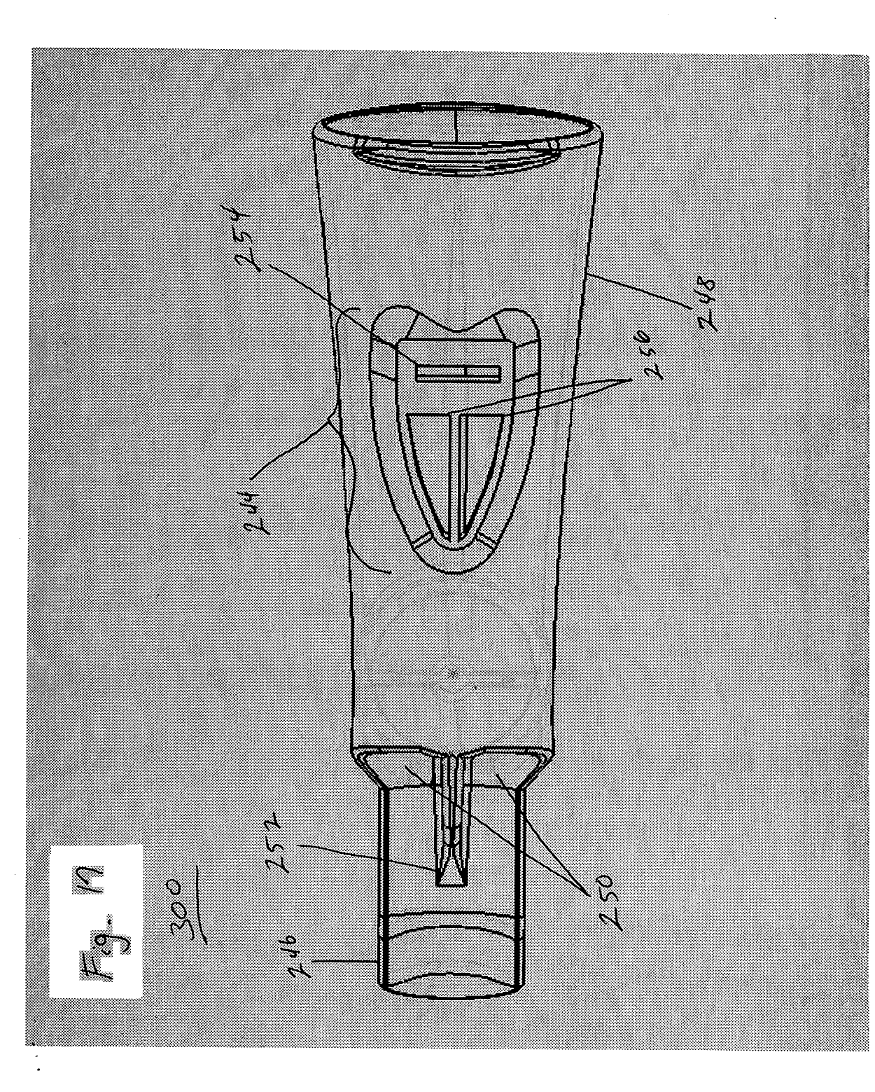

Referring to FIG. 16, a bottom view of the breath enhanced mouthpiece 300 is shown. Within the hollow body 232 is the baffle 240. The baffle 240 is attached to the interior of the mouthpiece 300 at a stop 258. The stop 258 is configured to control the degree of insertion of any fittings, including a nebulizer or aerosolization chamber, which may be attached to the mouthpiece 300 by sliding through the connection port 232 into the hollow body 230.

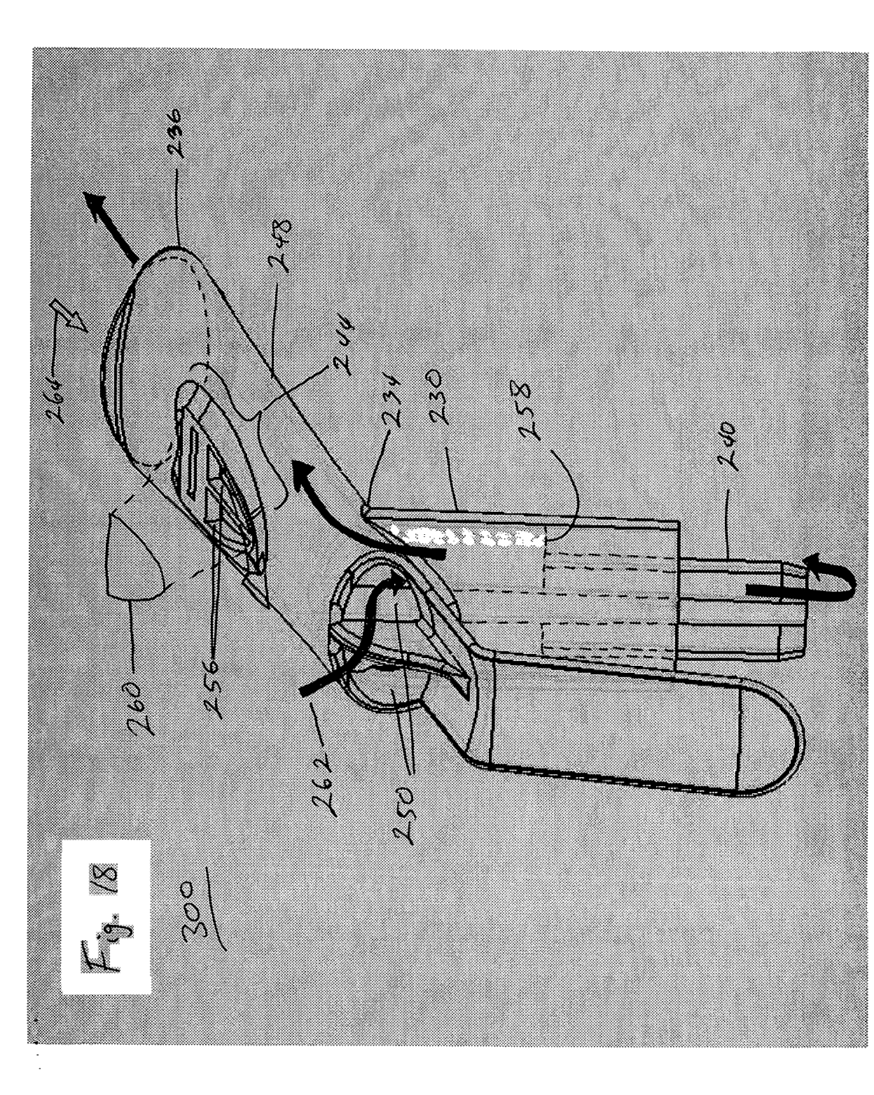

Referring to FIG. 17, a top view of the breath enhanced mouthpiece 300 is shown. The top of the breath enhanced mouthpiece 300 includes the exhaust vent structure 244. As shown, the recess 254 is a slot which is perpendicular to the long axis of the breath enhanced mouthpiece 300. Next to the recess 254 are the two outlets 256. It should be noted that the exhaust vent structure 244 may also be configured to receive fastening means for fastening a diaphragm thereto, such as a clip, pin, screw or adhesive.

Referring to FIG. 18, a rear view of the breath enhanced mouthpiece 300 is shown. A diaphragm 260 may be attached to the exhaust vent structure 244. The diaphragm 260 is configured to seal against the exhaust vent structure 244 in such a manner as to form a one-way valve. The diaphragm 260 and the exhaust structure 244 are configured to cooperate with one another to allow air to exit out the top of the breath enhanced mouthpiece 300 while preventing air from entering therethrough. Additionally, the exhaust vent structure 244 may be configured to be manually opened and closed by a user during operation.

Still referring to FIG. 18, an intake flow path 262 is represented by the solid arrows, and an exhaust flow path 264 is represented by the hollow arrows. The intake flow path 262 enters the breath enhanced mouthpiece 300 at inflow vent 250. After entering the inflow vent 250, the intake flow path 262 travels down the hollow body 230 towards the bottom of the baffle 240. At the bottom of the baffle 240 the intake flow path 262 makes an approximately 180 degree turn and proceeds up the hollow body 230 on the opposite side of the baffle 240 towards the union 234. At the union 234, the intake flow path 262 enters the drug delivery conduit 248 and proceeds towards the drug delivery port 236 to exit out of the breath enhanced mouthpiece 300 at the drug delivery port 236.

The exhaust flow path 264 enters the breath enhanced mouthpiece 300 at the drug delivery port 236. The exhaust flow path 264 travels along a portion of the drug delivery conduit 248 to exit out the top of the breath enhanced mouthpiece 300 through the outlets 256 of the exhaust structure 244.

The breath enhanced mouthpiece 10 is used to illustrate operation of the various embodiments of the invention. Referring back to FIG. 1, the breath enhanced mouthpiece 10 is attached to a nebulizer at the connection port 40. The nebulizer contains a supply of a drug or other substance which will be mixed or entrained in the air which passes through the aerosolization chamber area.

When a user puts the user interface or mouthpiece 18 into his mouth and inhales, air is drawn in through the intake 34 at the top of the hollow bodied 12 and travels down the baffle 42 shaped like a conduit and out its lower end. After exiting the baffle 42, the air flow enters the aerosolization chamber or nebulizer area where it is mixed with the drug or material to be delivered. The air now entrained with the drug or material to be delivered, passes out of the aerosolization area then travels back up the hollow body 12 along the exterior of the baffle 42. The air and drug or material combination then passes through the union inlet 46 and enters the drug delivery conduit 14. The air and drug or material then travel towards the drug delivery port 16. The air and drug or material then exit the breath enhanced mouthpiece 10 at the drug delivery port 16 and enters the user's mouth to find its way into the user's respiratory system.

The other embodiments work in a similar manner, with some of the embodiments relying on a diaphragm in combination with a vent or vents to direct air along certain flow paths. For example, the inlet vent 112 shown in FIG. 8 is configured so that air pressure caused by inhalation opens the inlet vent 112 and closes outlet vents 120. Thus inhalation causes the inlet vent 112 to allow air to enter the delivery flow path 130 and proceed towards the nebulization area and into the patient's mouth. During exhalation, air pressure causes the inlet vent 112 to close, and outlet vents 120 to open and allow air in the exhaust flow path 131 to exit the mouthpiece 100.

Note that although the above example illustrates vent operation by air pressure created during inhalation and exhalation, the vents may also be actuated directly by the user, such as through opening and closing the vents with his fingers. Additionally, the vents may be operated by any vent operation mechanism well know in the art, including, for example, electronic actuation, spring actuation, etc.

It should be noted that embodiments of the invention are of dimensions scaled to facilitate use by a variety of patients of different sizes and physical conditions. Thus, embodiments are typically sized to be easily grasped, easily inserted into, and easily held in a patient's mouth.

1. A drug delivery system, comprising:
a top flow path;
a conduit with an end in communication with the top flow path and an end configured to communicate with a nebulizer;
a connection port on the conduit configured to reversibly attach to a nebulizer;
a nebulizer in-flow path disposed within the conduit configured to communicate with a nebulizer; and
a nebulizer out-flow path disposed within the conduit configured to communicate with a nebulizer and the top flow path, wherein the nebulizer out-flow path is separate from the nebulizer in-flow path.

2. The drug delivery system of claim 1, wherein the top flow path is configured to direct a flow into a patient's lungs.

3. The drug delivery system of claim 2, wherein the top flow path further comprises a vent inlet configured to direct a flow into the top flow path.

4. The drug delivery system of claim 3, wherein the vent inlet is configured to be selectively sealable.

5. The drug delivery system of claim 1, wherein the nebulizer in-flow path is configured to direct flow into a nebulizer, and the nebulizer out-flow path is configured to direct flow out of a nebulizer.

6. The drug delivery system of claim 1, wherein the nebulizer in-flow path and the nebulizer out-flow path are coaxially disposed with respect to one another.

7. The drug delivery system of claim 1, wherein the connection port is configured to reversibly sealably attach to a nebulizer.

8. The drug delivery system of claim 7, wherein the nebulizer is configured to aerosolize a substance into a gas.

9. The drug delivery system of claim 8, wherein the gas comprises air.

10. The drug delivery system of claim 1, wherein the nebulizer in-flow path comprises an entrance configured to be selectively sealable.

11. A nebulizer mouthpiece, comprising:
a user interface having a delivery port;
a conduit connected to the user interface,
a connection port on the conduit, wherein the connection port is reversibly attachable to a corresponding fitting and the user interface and the connection port are configured to define a flow path between an aerosolization area and the delivery port;
an intake flow path configured to communicate with an aerosolization area; and
an exhaust flow path configured to communicate with an aerosolization area, wherein the exhaust flow path and the intake flow path are combined.

12. A drug delivery mouthpiece, comprising:
a hollow body with a top and a bottom;
an intake port and a delivery port proximate the top of the hollow body;
a reversibly attachable connection port proximate the bottom of the hollow body;
nebulizer inlet and a nebulizer outlet proximate the bottom of the hollow body; and
a baffle at least partially disposed within the hollow body, the hollow body and the baffle defining an air flow path having portions parallel to one another through an interior of the hollow body.

13. The drug delivery mouthpiece of claim 12, wherein the baffle defines an intake flow path between the intake port and the nebulizer inlet through the hollow body and a delivery flow path between the nebulizer outlet and the delivery port through the hollow body.

14. The drug delivery mouthpiece of claim 13, wherein the baffle comprises a conduit with the intake flow path disposed within the conduit and the delivery flow path disposed outside the conduit.

15. The drug delivery mouthpiece of claim 13, wherein the baffle comprises a substantially planar member disposed between the intake flow path and the delivery flow path.

16. The drug delivery mouthpiece of claim 13, wherein the connection port is configured to reversibly attach to a chamber.

17. The drug delivery mouthpiece of claim 16, wherein the baffle is configured to protrude into a chamber connected to the connection port.

18. The drug delivery mouthpiece of claim 17, wherein the baffle is configured to direct the intake flow path out of the hollow body and into a chamber connected to the connection port, and the baffle is configured to direct the delivery flow path from a chamber connected to the connection port into the hollow body.

19. The drug deliver mouthpiece of claim 12, wherein the delivery port is disposed on a conduit attached proximate a top of the hollow body.

20. The drug delivery system of claim 19, wherein the conduit comprises an exhaust outlet.

21. The drug delivery system of claim 20, wherein the exhaust outlet is selectable sealable.

22. The drug delivery system of claim 21, wherein the exhaust outlet comprises a one-way valve.

23. The drug delivery mouthpiece of claim 12, wherein the intake port is sealably configured.
